(19) United States
(12) Patent Application Publication
(10) Pub. No.: US 2023/0123169 A1
(43) Pub. Date: Apr. 20, 2023

(54) METHODS AND SYSTEMS FOR USE OF ANALYSIS ASSISTANT DURING ULTRASOUND IMAGING

(71) Applicant: GE Precision Healthcare LLC, Wauwatosa, WI (US)

(72) Inventor: Jurica {hacek over (S)}prem, Oslo (NO)

(21) Appl. No.: 17/502,712

(22) Filed: Oct. 15, 2021

(51) Int. Cl. G06T 7/00; A61B 8/00

(52) U.S. Cl. CPC G06T 7/0012; A61B 8/463; A61B 8/467; G06T 2207/20084; G06T 2207/10132; G06T 2207/30004

(57) ABSTRACT

Systems and methods are provided for use of analysis assistant during ultrasound imaging. Database may be acquired on a medical imaging technique during medical imaging based examination of a patient; and one or more medical images may be generated, based on the acquired dataset, and displayed. An end-result associated with at least one medical image may be provided, when triggered analysis assistance relating to the end-result associated with the at least one medical image may be provided. Providing the analysis assistance may include, automatically: identifying one or more intermediate steps performed or used in obtaining or determining the end-result; determining for each of the one or more intermediate steps corresponding information; and providing to a user the determined information associated with each of the one or more intermediate steps.

### FIELD

Aspects of the present disclosure relate to medical imaging solutions. More specifically, certain embodiments relate to methods and systems for use of analysis assistant during ultrasound imaging.

### BACKGROUND

Various medical imaging techniques may be used, such as in imaging organs and soft tissues in a human body. Examples of medical imaging techniques include ultrasound imaging, computed tomography (CT) scans, magnetic resonance imaging (MRI), etc. The manner by which images are generated during medical imaging depends on the particular technique.

For example, ultrasound imaging uses real time, non-invasive high frequency sound waves to produce ultrasound images, typically of organs, tissues, objects (e.g., fetus) inside the human body. Images produced or generated during medical imaging may be two-dimensional (2D), three-dimensional (3D), and/or four-dimensional (4D) images (essentially real-time/continuous 3D images). During medical imaging, imaging datasets (including, e.g., volumetric imaging datasets during 3D/4D imaging) are acquired and used in generating and rendering corresponding images (e.g., via a display) in real-time.

Existing medical imaging solutions may have some limitations. For example, in general existing medical imaging systems may only provide the end results associated with images, and related information (e.g., measurements) during medical imaging operations, but not any information relating to the how such end results may have been determined or calculated. Further limitations and disadvantages of conventional and traditional approaches will become apparent to one of skill in the art, through comparison of such systems with some aspects of the present disclosure, as set forth in the remainder of the present application with reference to the drawings.

### BRIEF SUMMARY

System and methods are provided for use of analysis assistant during ultrasound imaging, substantially as shown in and/or described in connection with at least one of the figures, as set forth more completely in the claims.

These and other advantages, aspects and novel features of the present disclosure, as well as details of one or more illustrated example embodiments thereof, will be more fully understood from the following description and drawings.

### BRIEF DESCRIPTION OF THE DRAWINGS

FIG. 1 is a block diagram illustrating an example medical imaging arrangement.

FIG. 2 is a block diagram illustrating an example ultrasound system.

FIGS. 3A-3C illustrate different end results that may be generated or obtained during medical imaging of lungs.

FIG. 4 illustrates an example use scenario based on medical imaging of the heart.

FIG. 5 illustrates a flowchart of an example process for utilizing analysis assistant during medical imaging.

### DETAILED DESCRIPTION

Certain implementations in accordance with the present disclosure may be directed to use of analysis assistant during ultrasound imaging. The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware Similarly, the programs may be stand-alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” as used in the context of ultrasound imaging is used to refer to an ultrasound mode such as B-mode (2D mode), M-mode, three-dimensional (3D) mode, CF-mode, PW Doppler, CW Doppler, MGD, and/or sub-modes of B-mode and/or CF such as Shear Wave Elasticity Imaging (SWEI), TVI, Angio, B-flow, BMI, BMI_Angio, and in some cases also MM, CM, TVD where the “image” and/or “plane” includes a single beam or multiple beams.

In addition, as used herein, the phrase “pixel” also includes embodiments where the data is represented by a “voxel.” Thus, both the terms “pixel” and “voxel” may be used interchangeably throughout this document.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC, or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. In addition, forming of images may be performed using channel combinations that may originate from more than one transmit event (e.g., synthetic aperture techniques).

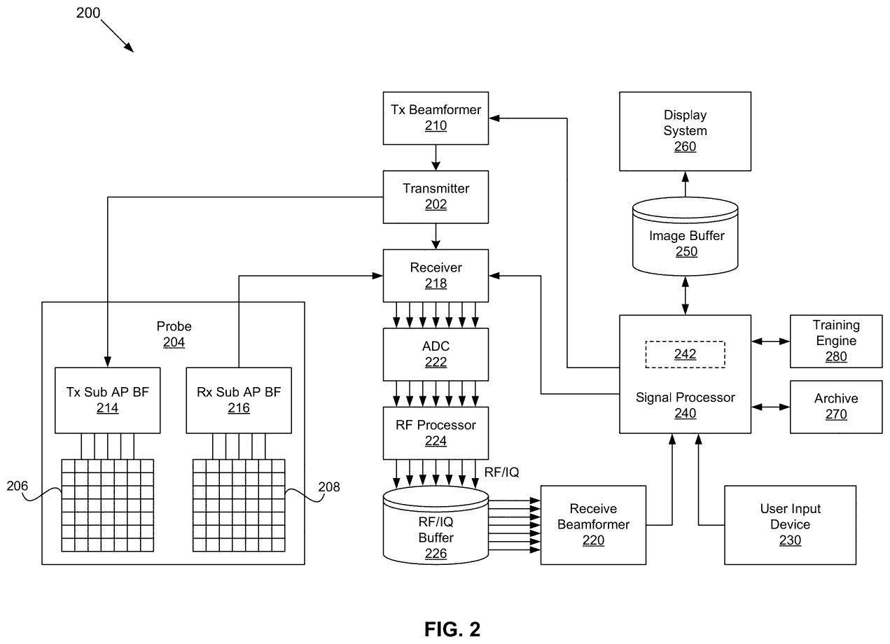

In various embodiments, processing to form images is performed in software, firmware, hardware, or a combination thereof. The processing may include use of beamforming. One example implementation of an ultrasound system having a software beamformer architecture formed in accordance with various embodiments as illustrated in FIG. 2.

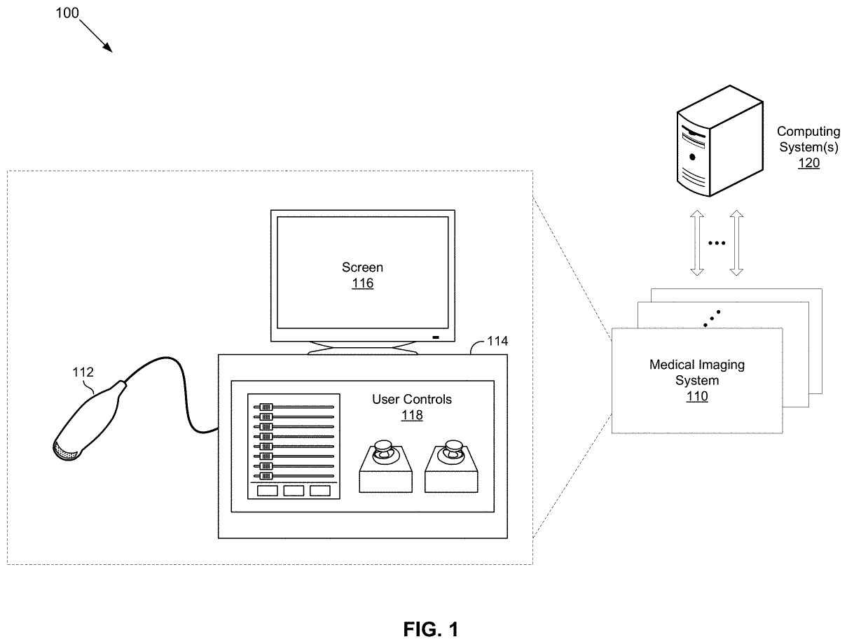

FIG. 1 is a block diagram illustrating an example medical imaging arrangement. Shown in FIG. 1 is an example medical imaging arrangement 100 that comprises one or more medical imaging systems 110 and one or more computing systems 120. The medical imaging arrangement 100 (including various elements thereof) may be configured to support medical imaging and solutions associated therewith.

The medical imaging system 110 comprise suitable hardware, software, or a combination thereof, for supporting medical imaging—that is enabling obtaining data used in generating and/or rendering images during medical imaging exams. Examples of medical imaging include ultrasound imaging, computed tomography (CT) scans, magnetic resonance imaging (MRI), etc. This may entail capturing of particular type of data, in particular manner, which may in turn be used in generating data for the images. For example, the medical imaging system 110 may be an ultrasound imaging system, configured for generating and/or rendering ultrasound images. An example implementation of an ultrasound system, which may correspond to the medical imaging system 110, is described in more detail with respect to FIG. 2.

As shown in FIG. 1, the medical imaging system 110 may comprise a scanner device 112, which may be portable and movable, and a display/control unit 114. The scanner device 112 may be configured for generating and/or capturing particular type of imaging signals (and/or data corresponding thereto), such as by being moved over a patient's body (or part thereof), and may comprise suitable circuitry for performing and/or supporting such functions. The scanner device 112 may be an ultrasound probe, MRI scanner, CT scanner, or any suitable imaging device. For example, where the medical imaging system 110 is an ultrasound system, the scanner device 112 may emit ultrasound signals and capture echo ultrasound images.

The display/control unit 114 may be configured for displaying images (e.g., via a screen 116). In some instances, the display/control unit 114 may further be configured for generating the displayed images, at least partly. Further, the display/control unit 114 may also support user input/output. For example, the display/control unit 114 may provide (e.g., via the screen 116), in addition to the images, user feedback (e.g., information relating to the system, functions thereof, settings thereof, etc.). The display/control unit 114 may also support user input (e.g., via user controls 118), such as to allow controlling of the medical imaging. The user input may be directed to controlling display of images, selecting settings, specifying user preferences, requesting feedback, etc.

In some implementations, the medical imaging arrangement 100 may also incorporate additional and dedicated computing resources, such as the one or more computing systems 120. In this regard, each computing system 120 may comprise suitable circuitry, interfaces, logic, and/or code for processing, storing, and/or communication data. The computing system 120 may be dedicated equipment configured particularly for use in conjunction with medical imaging, or it may be a general purpose computing system (e.g., personal computer, server, etc.) set up and/or configured to perform the operations described hereinafter with respect to the computing system 120. The computing system 120 may be configured to support operations of the medical imaging systems 110, as described below. In this regard, various functions and/or operations may be offloaded from the imaging systems. This may be done to streamline and/or centralize certain aspects of the processing, to reduce cost—e.g., by obviating the need to increase processing resources in the imaging systems.

The computing systems 120 may be set up and/or arranged for use in different ways. For example, in some implementations a single computing system 120 may be used; in other implementations multiple computing systems 120, either configured to work together (e.g., based on distributed-processing configuration), or separately, with each computing system 120 being configured to handle particular aspects and/or functions, and/or to process data only for particular medical imaging systems 110. Further, in some implementations, the computing systems 120 may be local (e.g., co-located with one or more medical imaging systems 110, such within the same facility and/or same local network); in other implementations, the computing systems 120 may be remote and thus can only be accessed via remote connections (e.g., via the Internet or other available remote access techniques). In a particular implementation, the computing systems 120 may be configured in cloud-based manner, and may be accessed and/or used in substantially similar way that other cloud-based systems are accessed and used.

Once data is generated and/or configured in the computing system 120, the data may be copied and/or loaded into the medical imaging systems 110. This may be done in different ways. For example, the data may be loaded via directed connections or links between the medical imaging systems 110 and the computing system 120. In this regard, communications between the different elements in the medical imaging arrangement 100 may be done using available wired and/or wireless connections, and/or in accordance any suitable communication (and/or networking) standards or protocols. Alternatively, or additionally, the data may be loaded into the medical imaging systems 110 indirectly. For example, the data may be stored into suitable machine readable media (e.g., flash card, etc.), which are then used to load the data into the medical imaging systems 110 (on-site, such as by users of the systems (e.g., imaging clinicians) or authorized personnel), or the data may be downloaded into local communication-capable electronic devices (e.g., laptops, etc.), which are then used on-site (e.g., by users of the systems or authorized personnel) to upload the data into the medical imaging systems 110, via direct connections (e.g., USB connector, etc.).

In operation, the medical imaging system 110 may be used in generating and presenting (e.g., rendering or displaying) images during medical exams, and/or in supporting user input/output in conjunction therewith. The images may be 2D, 3D, and/or 4D images. The particular operations or functions performed in the medical imaging system 110 to facilitate the generating and/or presenting of images depends on the type of system—that is, the manner by which the data corresponding to the images is obtained and/or generated. For example, in computed tomography (CT) scans based imaging, the data is based on emitted and captured x-rays signals. In ultrasound imaging, the data is based on emitted and echo ultrasound signals. This described in more details with respect to the example ultrasound-based implementation illustrated in and described with respect to FIG. 2.

In various implementations in accordance with the present disclosure, medical imaging systems and/or architectures (e.g., the medical imaging system 110 and/or the medical imaging arrangement 100 as a whole) may be configured to support implementing and using use of analysis assistant during medical imaging operations (e.g., ultrasound imaging). In this regard, many medical imaging solutions may incorporate tools for handling certain tasks or functions during medical imaging operations, such as performing particular measurements based on and relating to obtained medical images. Such tools may be configured to incorporate advanced processing techniques, such as artificial intelligence (AI) based processing, in performing at least some of tasks or functions associated therewith. Most of the tools may use intermediate steps (e.g., image segmentations, intermediate measurements, classification, etc.) in order to reach the end result. The intermediate steps may be in particular order (e.g., sequential order), or may be in any order—that is, the intermediate steps (or at least some of them) may be need to be performed but without any inter-step dependency thus obviating the need for particular step(s) to proceed other step(s).

These intermediate steps may be of clinical interest to some users (e.g., operators of the system, doctors or specialists reviewing the medical images, etc.). For example, during some imaging operations, clinical values corresponding to intermediate steps, such as image segmentations that may be used to calculate the ejection fraction of a heart for example, may be of clinical interest to the users. Current tools (conventional tools and/or advanced processing (e.g., AI) based tools) may only provide the end result needed for such measurements obtained or determined by such tools, but may not provide any information relating to any intermediate steps performed by the tools. Implementations in accordance with present disclosure mitigates some of the shortcomings and limitations of existing solutions, by incorporating mechanisms for providing information relating to intermediate steps. For such tools, analysis assistant based solutions implemented in accordance with the present disclosure may provide the users with all the additional information on how the final measurement was obtained, for both AI and conventional tools or the entire pipelines of different tools that are used by user in medical imaging setups.

In particular, in various embodiments medical imaging systems and/or setups (including but not limited to ultrasound based systems or setups) may incorporate analysis assistant as an automatic tool configured to provide users an overview showing intermediate steps of a selected tool's pipeline (e.g., measurement tool used for obtaining particular measurements during and/or based on medical imaging) together with intermediate results (e.g., of AI algorithms that are used within the selected tool). For example, the user(s) may be provided with all the intermediate steps and additional interactivity options (e.g., manual changes of the results within the tool's pipeline). The solutions and example implementations associated therewith are described in more details below.

FIG. 2 is a block diagram illustrating an example ultrasound imaging system. Shown in FIG. 2 is an ultrasound imaging system 200, which may be configured to support use of analysis assistant during ultrasound imaging in accordance with the present disclosure.

The ultrasound imaging system 200 may be configured for providing ultrasound imaging, and as such may comprise suitable circuitry, interfaces, logic, and/or code for performing and/or supporting ultrasound imaging related functions. The ultrasound imaging system 200 may correspond to the medical imaging system 110 of FIG. 1. The ultrasound imaging system 200 comprises, for example, a transmitter 202, an ultrasound probe 204, a transmit beamformer 210, a receiver 218, a receive beamformer 220, a RF processor 224, a RF/IQ buffer 226, a user input module 230, a signal processor 240, an image buffer 250, a display system 260, an archive 270, and a training engine 280.

The transmitter 202 may comprise suitable circuitry, interfaces, logic, and/or code that may be operable to drive an ultrasound probe 204. The ultrasound probe 204 may comprise a two dimensional (2D) array of piezoelectric elements. The ultrasound probe 204 may comprise a group of transmit transducer elements 206 and a group of receive transducer elements 208, that normally constitute the same elements. In certain embodiment, the ultrasound probe 204 may be operable to acquire ultrasound image data covering at least a substantial portion of an anatomy, such as the heart, a blood vessel, or any suitable anatomical structure.

The transmit beamformer 210 may comprise suitable circuitry, interfaces, logic, and/or code that may be operable to control the transmitter 202 which, through a transmit sub-aperture beamformer 214, drives the group of transmit transducer elements 206 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 208.

The group of receive transducer elements 208 in the ultrasound probe 204 may be operable to convert the received echoes into analog signals, undergo sub-aperture beamforming by a receive sub-aperture beamformer 216 and are then communicated to a receiver 218. The receiver 218 may comprise suitable circuitry, interfaces, logic, and/or code that may be operable to receive the signals from the receive sub-aperture beamformer 216. The analog signals may be communicated to one or more of the plurality of A/D converters 222.

The plurality of A/D converters 222 may comprise suitable circuitry, interfaces, logic, and/or code that may be operable to convert the analog signals from the receiver 218 to corresponding digital signals. The plurality of A/D converters 222 are disposed between the receiver 218 and the RF processor 224. Notwithstanding, the disclosure is not limited in this regard. Accordingly, in some embodiments, the plurality of A/D converters 222 may be integrated within the receiver 218.

The RF processor 224 may comprise suitable circuitry, interfaces, logic, and/or code that may be operable to demodulate the digital signals output by the plurality of A/D converters 222. In accordance with an embodiment, the RF processor 224 may comprise a complex demodulator (not shown) that is operable to demodulate the digital signals to form I/Q data pairs that are representative of the corresponding echo signals. The RF or I/Q signal data may then be communicated to an RF/IQ buffer 226. The RF/IQ buffer 226 may comprise suitable circuitry, interfaces, logic, and/or code that may be operable to provide temporary storage of the RF or I/Q signal data, which is generated by the RF processor 224.

The receive beamformer 220 may comprise suitable circuitry, interfaces, logic, and/or code that may be operable to perform digital beamforming processing to, for example, sum the delayed channel signals received from RF processor 224 via the RF/IQ buffer 226 and output a beam summed signal. The resulting processed information may be the beam summed signal that is output from the receive beamformer 220 and communicated to the signal processor 240. In accordance with some embodiments, the receiver 218, the plurality of A/D converters 222, the RF processor 224, and the beamformer 220 may be integrated into a single beamformer, which may be digital. In various embodiments, the ultrasound imaging system 200 comprises a plurality of receive beamformers 220.

The user input device 230 may be utilized to input patient data, scan parameters, settings, select protocols and/or templates, interact with an artificial intelligence segmentation processor to select tracking targets, and the like. In an example embodiment, the user input device 230 may be operable to configure, manage and/or control operation of one or more components and/or modules in the ultrasound imaging system 200. In this regard, the user input device 230 may be operable to configure, manage and/or control operation of the transmitter 202, the ultrasound probe 204, the transmit beamformer 210, the receiver 218, the receive beamformer 220, the RF processor 224, the RF/IQ buffer 226, the user input device 230, the signal processor 240, the image buffer 250, the display system 260, archive 270, and/or the training engine 280.

For example, the user input device 230 may include button(s), rotary encoder(s), a touchscreen, motion tracking, voice recognition, a mouse device, keyboard, camera and/or any other device capable of receiving user directive(s). In certain embodiments, one or more of the user input devices 230 may be integrated into other components, such as the display system 260 or the ultrasound probe 204, for example.

As an example, user input device 230 may include a touchscreen display. As another example, user input device 230 may include an accelerometer, gyroscope, and/or magnetometer attached to and/or integrated with the probe 204 to provide gesture motion recognition of the probe 204, such as to identify one or more probe compressions against a patient body, a pre-defined probe movement or tilt operation, or the like. In some instances, the user input device 230 may include, additionally or alternatively, image analysis processing to identify probe gestures by analyzing acquired image data. In accordance with the present disclosure, the user input and functions related thereto may be configured to support use of new data storage scheme, as described in this disclosure. For example, the user input device 230 may be configured to support receiving user input directed at triggering and managing (where needed) application of separation process, as described herein, and /or to provide or set parameters used in performing such process. Similarly, the user input device 230 may be configured to support receiving user input directed at triggering and managing (where needed) application of the recovery process, as described herein, and/or to provide or set parameters used in performing such process.

The signal processor 240 may comprise suitable circuitry, interfaces, logic, and/or code that may be operable to process ultrasound scan data (i.e., summed IQ signal) for generating ultrasound images for presentation on a display system 260. The signal processor 240 is operable to perform one or more processing operations according to a plurality of selectable ultrasound modalities on the acquired ultrasound scan data. In an example embodiment, the signal processor 240 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 226 during a scanning session and processed in less than real-time in a live or off-line operation. In various embodiments, the processed image data can be presented at the display system 260 and/or may be stored at the archive 270.

The archive 270 may be a local archive, a Picture Archiving and Communication System (PACS), or any suitable device for storing images and related information, or may be coupled to such device or system for facilitating the storage and/or achieving of the imaging related data. In an example implementation, the archive 270 is further coupled to a remote system such as a radiology department information system, hospital information system, and/or to an internal or external network (not shown) to allow operators at different locations to supply commands and parameters and/or gain access to the image data.

The signal processor 240 may be one or more central processing units, microprocessors, microcontrollers, and/or the like. The signal processor 240 may be an integrated component, or may be distributed across various locations, for example. The signal processor 240 may be configured for receiving input information from the user input device 230 and/or the archive 270, generating an output displayable by the display system 260, and manipulating the output in response to input information from the user input device 230, among other things. The signal processor 240 may be capable of executing any of the method(s) and/or set(s) of instructions discussed herein in accordance with the various embodiments, for example.

The ultrasound imaging system 200 may be operable to continuously acquire ultrasound scan data at a frame rate that is suitable for the imaging situation in question. Typical frame rates range from 20-220 but may be lower or higher. The acquired ultrasound scan data may be displayed on the display system 260 at a display-rate that can be the same as the frame rate, or slower or faster. The image buffer 250 is included for storing processed frames of acquired ultrasound scan data that are not scheduled to be displayed immediately. Preferably, the image buffer 250 is of sufficient capacity to store at least several minutes' worth of frames of ultrasound scan data. The frames of ultrasound scan data are stored in a manner to facilitate retrieval thereof according to its order or time of acquisition. The image buffer 250 may be embodied as any known data storage medium.

In an example embodiment, the signal processor 240 may comprise an analysis assistant module 242, which comprises suitable circuitry, interfaces, logic, and/or code that may be configured to perform and/or support various functions or operations relating to, or in support of use of analysis assistant during ultrasound imaging, as described in this disclosure.

In some implementations, the signal processor 240 (and/or components thereof, such as the analysis assistant module 242) may be configured to implement and/or use artificial intelligence and/or machine learning techniques to enhance and/or optimize imaging related functions or operations. For example, the signal processor 240 (and/or components thereof, such as the analysis assistant module 242) may be configured to implement and/or use deep learning techniques and/or algorithms, such as by use of deep neural networks (e.g., a convolutional neural network (CNN)), and/or may utilize any suitable form of artificial intelligence based processing techniques or machine learning processing functionality (e.g., for image analysis). Such artificial intelligence based image analysis may be configured to, e.g., analyze acquired ultrasound images, such as to identify, segment, label, and track structures (or tissues thereof) meeting particular criteria and/or having particular characteristics.

In an example implementation, the signal processor 240 (and/or components thereof, such as the analysis assistant module 242) may be provided as a deep neural network, which may be made up of, for example, an input layer, an output layer, and one or more hidden layers in between the input and output layers. Each of the layers may be made up of a plurality of processing nodes that may be referred to as neurons.

For example, the deep neural network may include an input layer having a neuron for each pixel or a group of pixels from a scan plane of an anatomical structure, and the output layer may have a neuron corresponding to a plurality of pre-defined structures or types of structures (or tissue(s) therein). Each neuron of each layer may perform a processing function and pass the processed ultrasound image information to one of a plurality of neurons of a downstream layer for further processing. As an example, neurons of a first layer may learn to recognize edges of structure in the ultrasound image data. The neurons of a second layer may learn to recognize shapes based on the detected edges from the first layer. The neurons of a third layer may learn positions of the recognized shapes relative to landmarks in the ultrasound image data. The neurons of a fourth layer may learn characteristics of particular tissue types present in particular structures, etc. Thus, the processing performed by the deep neural network (e.g., convolutional neural network (CNN)) may allow for identifying biological and/or artificial structures in ultrasound image data with a high degree of probability.

In some implementations, the signal processor 240 (and/or components thereof, such as the analysis assistant module 242) may be configured to perform or otherwise control at least some of the functions performed thereby based on a user instruction via the user input device 230. As an example, a user may provide a voice command, probe gesture, button depression, or the like to issue a particular instruction, such as to initiate and/or control various aspects of the new data management scheme, including artificial intelligence (AI) based operations, and/or to provide or otherwise specify various parameters or settings relating thereto, as described in this disclosure.

The training engine 280 may comprise suitable circuitry, interfaces, logic, and/or code that may be operable to train the neurons of the deep neural network(s) of the signal processor 240 (and/or components thereof, such as the analysis assistant module 242). For example, the signal processor 240 may be trained to identify particular structures and/or tissues (or types thereof) provided in an ultrasound scan plane, with the training engine 280 training the deep neural network(s) thereof to perform some of the required functions, such as using databases(s) of classified ultrasound images of various structures.

As an example, the training engine 280 may be configured to utilize ultrasound images to train the signal processor 240 (and/or components thereof, such as the analysis assistant module 242), such as based on particular structure(s) and/or characteristics thereof, particular tissues and/or characteristics thereof, etc. For example, with the respect to structure(s), the training engine 280 may be configured to identify and utilize such characteristics as appearance of structure edges, appearance of structure shapes based on the edges, positions of the shapes relative to landmarks in the ultrasound image data, and the like. In various embodiments, the databases of training images may be stored in the archive 270 or any suitable data storage medium. In certain embodiments, the training engine 280 and/or training image databases may be external system(s) communicatively coupled via a wired or wireless connection to the ultrasound imaging system 200.

In operation, the ultrasound imaging system 200 may be used in generating ultrasonic images, including two-dimensional (2D), three-dimensional (3D), and/or four-dimensional (4D) images. In this regard, the ultrasound imaging system 200 may be operable to continuously acquire ultrasound scan data at a particular frame rate, which may be suitable for the imaging situation in question. For example, frame rates may range from 30-70 but may be lower or higher. The acquired ultrasound scan data may be displayed on the display system 260 at a display-rate that can be the same as the frame rate, or slower or faster. An image buffer 250 is included for storing processed frames of acquired ultrasound scan data not scheduled to be displayed immediately. Preferably, the image buffer 250 is of sufficient capacity to store at least several seconds' worth of frames of ultrasound scan data. The frames of ultrasound scan data are stored in a manner to facilitate retrieval thereof according to its order or time of acquisition. The image buffer 250 may be embodied as any known data storage medium.

In some instances, the ultrasound imaging system 200 may be configured to support grayscale and color based operations. For example, the signal processor 240 may be operable to perform grayscale B-mode processing and/or color processing. The grayscale B-mode processing may comprise processing B-mode RF signal data or IQ data pairs. For example, the grayscale B-mode processing may enable forming an envelope of the beam-summed receive signal by computing the quantity (I²+Q²)^{1/2}. The envelope can undergo additional B-mode processing, such as logarithmic compression to form the display data.

The display data may be converted to X-Y format for video display. The scan-converted frames can be mapped to grayscale for display. The B-mode frames that are provided to the image buffer 250 and/or the display system 260. The color processing may comprise processing color based RF signal data or IQ data pairs to form frames to overlay on B-mode frames that are provided to the image buffer 250 and/or the display system 260. The grayscale and/or color processing may be adaptively adjusted based on user input—e.g., a selection from the user input device 230, for example, for enhance of grayscale and/or color of particular area.

In some instances, ultrasound imaging may include generation and/or display of volumetric ultrasound images—that is where objects (e.g., organs, tissues, etc.) are displayed three-dimensional 3D. In this regard, with 3D (and similarly 4D) imaging, volumetric ultrasound datasets may be acquired, comprising voxels that correspond to the imaged objects. This may be done, e.g., by transmitting the sound waves at different angles rather than simply transmitting them in one direction (e.g., straight down), and then capture their reflections back. The returning echoes (of transmissions at different angles) are then captured, and processed (e.g., via the signal processor 240) to generate the corresponding volumetric datasets, which may in turn be used in creating and/or displaying volume (e.g. 3D) images, such as via the display 250. This may entail use of particular handling techniques to provide the desired 3D perception.

For example, volume rendering techniques may be used in displaying projections (e.g., 3D projections) of the volumetric (e.g., 3D) datasets. In this regard, rendering a 3D projection of a 3D dataset may comprise setting or defining a perception angle in space relative to the object being displayed, and then defining or computing necessary information (e.g., opacity and color) for every voxel in the dataset. This may be done, for example, using suitable transfer functions for defining RGBA (red, green, blue, and alpha) value for every voxel.

In some embodiments, the ultrasound imaging system 200 may be configured to support solutions in accordance with the present disclosure, such as by incorporating components and/or functions for facilitating and supporting use of analysis assistant during ultrasound imaging. For example, the ultrasound imaging system 200 may incorporate the analysis assistant module 242, which may be configured to implement and perform the analysis assistant related functions as described herein. Alternatively or additionally, at least a portion of the analysis assistant related functions may be offloaded to an external system (e.g., local dedicated computing system, remote (e.g., Cloud-based) server, etc.). In this regard, ultrasound imaging system 200 may incorporate tools for handling certain tasks or functions during ultrasound imaging operations, such as performing particular measurements based on and relating to obtained ultrasound images. Such tools may be configured to use or rely on convention processing and/or advanced processing, such as artificial intelligence (AI) based processing, in performing at least some of tasks or functions associated therewith. For example, the processor 240 may be configured to execute such tools, or at least some of the functions or processing (conventional and/or AI-based) associated therewith. These tools typically may only provide the end result for certain measurements. However, in some instances, the tools may actually use intermediate steps (e.g., image segmentations, intermediate measurements, classification, etc.) in order to reach the end results that are provided to the users (e.g., operators of the system, doctors or specialists reviewing the ultrasound images, etc.). In this regard, as noted above such intermediate steps may need to be performed in particular order (e.g., sequential order), or may performed in any order.

In some instances, such intermediate steps may be of clinical interest to the users (e.g., operators of the system, doctors or specialists reviewing the ultrasound images, etc.). For example, during ultrasound imaging of the heart, clinical values corresponding to intermediate steps, such as image segmentations that may be used to calculate the ejection fraction of a heart for example, may be of clinical interest to the users. Providing information relating to such intermediate steps may be advantageous. For example, users may find it difficult to accept only the final result from tools (conventional tools and/or advanced processing (e.g., AI) based tools) without knowing how the end result was obtained by such tools. In order to provide clearer understanding on how the tools work and provide good quality results, it is of great benefit to provide such information to the user upon request. Further, some tools may incorporate or use plurality of different smaller tools (e.g., configured to run or executed sequentially, in parallel, in any order, or any in combination thereof) within a large pipeline, and as such results from each of these smaller tools need to be accurate for the overall end result to be reliable. Thus, it would be very beneficial if users could investigate which of these tools are the ones that might have influenced the final end result of the tool used. Users could be offered to correct only a part of the whole pipeline in order to make their clinical assessments with greater confidence.

However, in existing solutions such tools (conventional and/or AI-based tools) may only provide the end results (e.g., measurements) obtained or determined by such tools, but may not provide any information relating to any intermediate steps performed by the tools. Therefore, use of analysis assistant, in accordance with the present disclosure may provide the users with the additional information relating to such intermediate steps—e.g., the intermediate heat maps or locations within the image where the tool focused its analysis and how the tool determined and reached the final end results provided to the user during the analysis. Accordingly, use of the analysis assistant may enhance and/or ensure explainability and interpretability of the tools to the users together with other tools within the bigger pipeline.

In some implementations, advanced processing techniques, such as artificial intelligence (AI) or other machine learning based techniques may be used in conjunction with the assistant analysis functions performed in the system. In this regard, the ultrasound imaging system 200, particularly via the processor 240 (and/or components thereof, such as the analysis assistant module 242) may be configured to implement and/or support use of artificial intelligence (AI) based techniques in conjunction with the assistant analysis based solutions. For example, the analysis assistant module 242 (e.g., in conjunction with the archive 270 and/or the training engine 280) may be configured to support and use of artificial intelligence (AI) based processing when performing some the tasks or functions relating to the analysis assistant, such as identifying the pertinent intermediate steps, assessing the order of the intermediate steps (if any), assessing utilized smaller tools (if any) obtaining or generating information associated with each of the intermediate steps, generating or otherwise effecting the providing of information relating to the intermediate (including, e.g., incorporating at least some of the information into generated/displayed images), and the like.

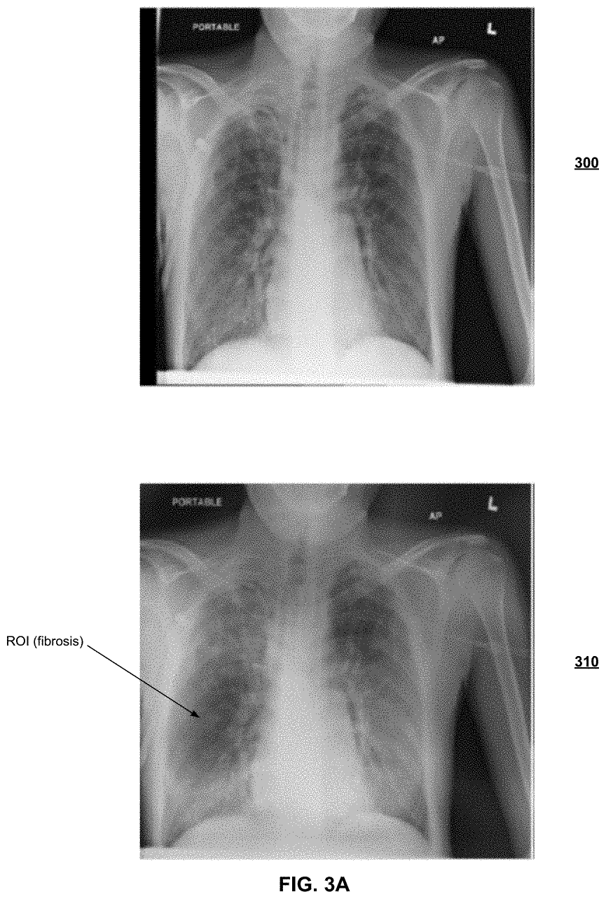

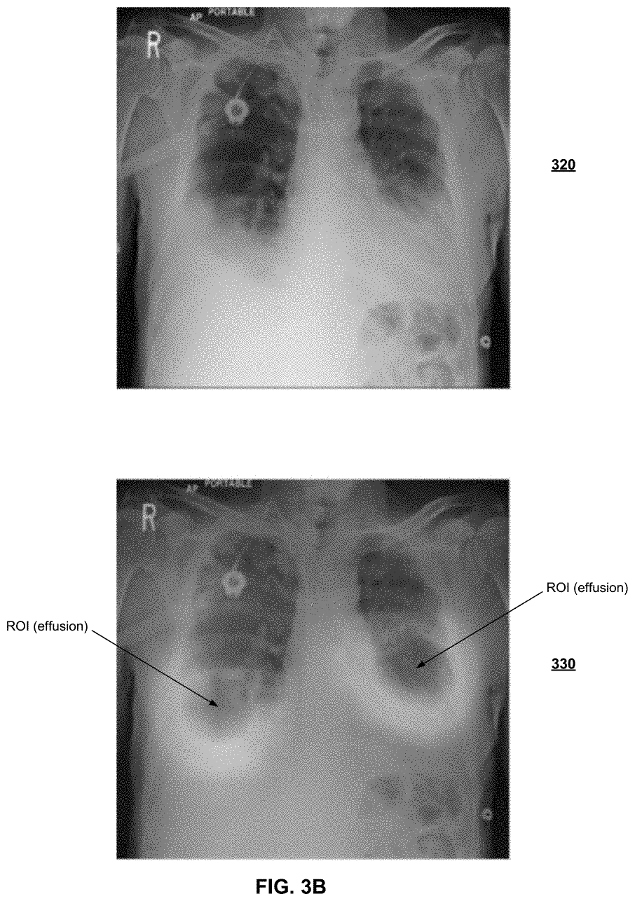

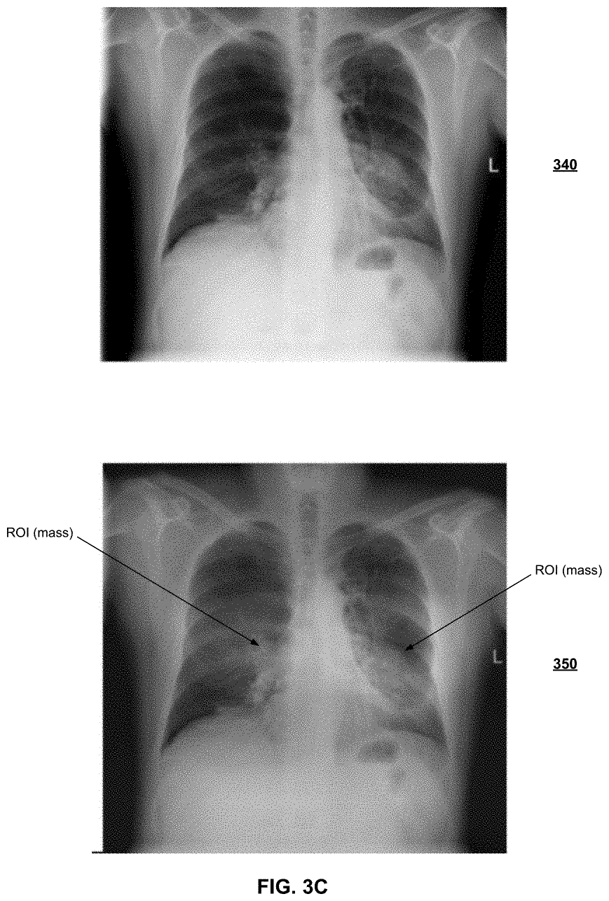

FIGS. 3A-3C illustrate different end results that may be generated or obtained during medical imaging of lungs. In this regard, each of the end results shown in FIGS. 3A-3C may be obtained by use or application of particular tool or function in suitable medical imaging system (e.g., the ultrasound system 200 of FIG. 2). For example, shown in FIG. 3A shown is image (or screenshot thereof) 310 corresponding to end result from applying of tool for identifying in a medical image 300 (e.g., ultrasound image) of region(s) of interest (ROIs) that may indicate presence of fibrosis in the lungs. Similarly, shown in FIG. 3B shown is image (or screenshot thereof) 310 corresponding to end result from applying of tool for identifying in a medical image 300 (e.g., ultrasound image) of region(s) of interest (ROIs) that may indicate presence of effusion in the lungs. Further, shown in FIG. 3C shown is image (or screenshot thereof) 310 corresponding to end result from applying of tool for identifying in a medical image 300 (e.g., ultrasound image) of region(s) of interest (ROIs) that may indicate presence of mass (e.g., tumor or the like) in the lungs.

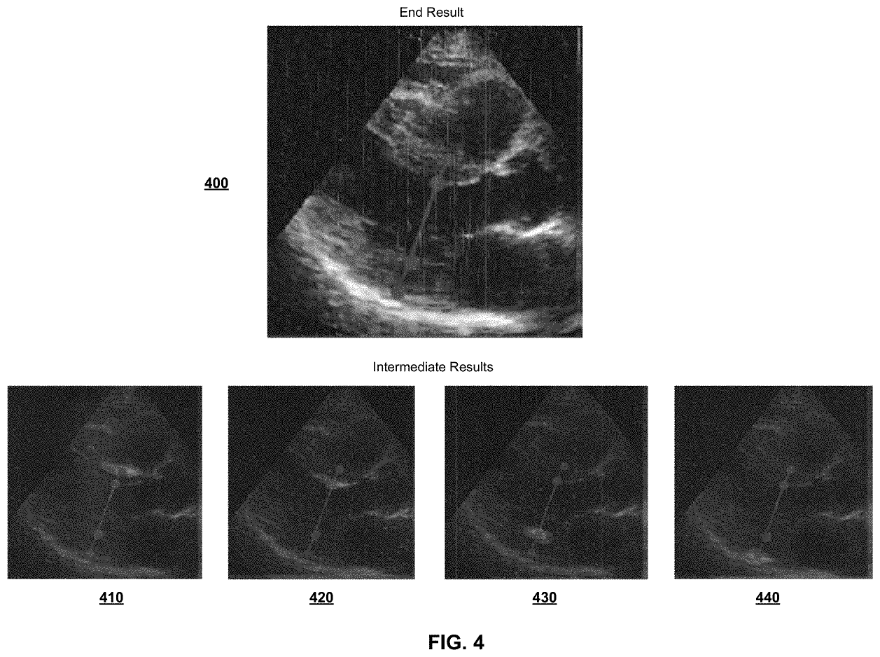

While these end results (or images corresponding thereto) are useful, in some instances may be interested in viewing images of, and/or obtaining information relating to any intermediate steps in the tools or functions used in generating or obtaining these end results. The analysis assistant tool implemented in the present disclosure may allow for that. In this regard, when activated or triggered (e.g., manually based on user input, automatically such as based on predefined criteria or requirement, etc.), the analysis assistant tool may determine when such intermediate steps may exist, and if so may obtain information related to these intermediate steps and provide that information, such as by displaying images corresponding to each of the intermediate steps. This may allow users to assess and confirm the end results as provided. An example use scenario with such intermediate steps is shown and described with respect to FIG. 4.

FIG. 4 illustrates an example use scenario based on medical imaging of the heart. Shown in FIG. 4 is screenshot 400 of a medical image (e.g., ultrasound image) displayed via a suitable medical imaging system (e.g., the ultrasound system 200 of FIG. 2). In accordance with the present disclosure, images corresponding to various intermediate results may also be displayed, such as in response to user input for triggering the analysis assistant tool. For example, in the use case illustrated in FIG. 4, images (410, 420, 430 and 440) corresponding to intermediate steps of intact ventricular septum (IVS), left ventricular internal dimension (LVID), left ventricular posterior wall (LVPW), which may be done twice. These intermediate steps may be in any order-—that is, the steps need not to be performed in particular order. The information corresponding to the intermediate steps may be determined, obtained, and/or generated (e.g., by the analysis assistant module 242 in the ultrasound system 200 of FIG. 2), and the corresponding images 410, 420, 430 and 440 may be generated and displayed to the user. This may allow the user to assess each of the intermediate steps, separately, which in turn may allow for assessing the end result as a whole.

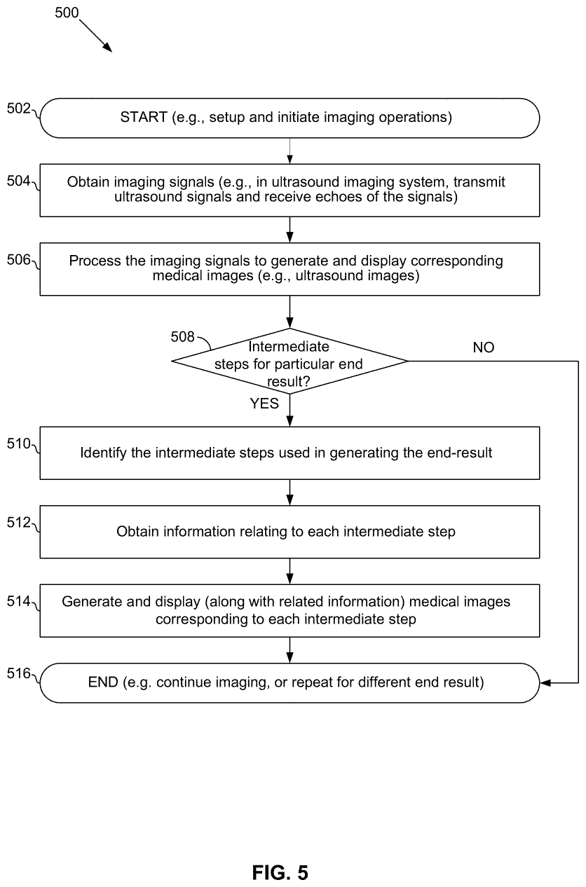

FIG. 5 illustrates a flowchart of an example process for utilizing analysis assistant during medical imaging. Shown in FIG. 5 is flow chart 500, comprising a plurality of example steps (represented as blocks 502-514), which may be performed in a suitable system (e.g., the medical imaging system 110 of FIG. 1, the ultrasound imaging system 200 of FIG. 2, etc.) for utilizing analysis assistant during medical imaging operations.

In start step 502, the system may be setup, and operations may initiate.

In step 504, imaging signals may be obtained. For example, in ultrasound imaging system, this may comprise transmitting ultrasound signals and receiving/capturing echoes of the signals).

In step 506, the imaging signals may be processed, to generate and display corresponding medical images (e.g., ultrasound images).

In step 508, it may be determined whether (or not) intermediate steps for particular end result are requested or needed. This may be done in response to user input (e.g., command or selection for triggering analysis assistant, based on pre-defined/pre-set triggering criteria, etc.). In instances where intermediate steps are not requested, the process may jump to end step 516; otherwise, the process may proceed to step 510.

In step 510, intermediate steps used in generating the end-result may be identified. This may be based on pre-programmed information, based on determining of any smaller tools used, etc.

In step 512, information relating to each intermediate step may be obtained.

In step 514, medical images corresponding to each intermediate step may be generated and displayed (along with related information).

The process may terminate in end step 516. In this regard, the ending may comprise continuing imaging operations, repeating the processing for a different end-result (e.g., steps 508-514), or simply terminating all imaging operations.

An example method, for use of analysis assistant, in accordance with the present disclosure, comprises acquiring dataset based on a medical imaging technique during medical imaging based examination of a patient; generating one or more medical images based on the acquired dataset; displaying the one or more medical images via a display device; providing an end-result associated with at least one medical image; and providing analysis assistance relating to the end-result associated with the at least one medical image, wherein providing the analysis assistance comprises, automatically: identifying one or more intermediate steps performed or used in obtaining or determining the end-result; determining for each of the one or more intermediate steps corresponding information; and providing to a user the determined information associated with each of the one or more intermediate steps.

In an example embodiment, the method further comprises providing the analysis assistance in response to at least one trigger, wherein the at least one trigger comprises at least one of user input or pre-defined condition.

In an example embodiment, the method further comprises performing at least some of the functions associated with providing the analysis assistance using artificial intelligence (AI) based processing.

In an example embodiment, the method further comprises providing the determined information associated with at least one intermediate step in conjunction with display at least one corresponding image.

In an example embodiment, providing the determined information associated with the at least one intermediate step further comprises: generating at least one medical image based on the acquired dataset corresponding to the at least one intermediate step; adjusting the at least one medical image to incorporate at least a portion of the determined information, or visual indication corresponding to the determined information; and displaying the at least one medical image via the display device.

In an example embodiment, the method further comprises providing the end-result associated with the at least one medical image in response to at least one trigger, wherein the at least one trigger comprises a tool that is executed in response to at least one of user input and pre-defined condition.

In an example embodiment, the tool comprises an artificial intelligence (AI) based tool.

An example non-transitory computer readable medium in accordance with the present disclosure has stored thereon a computer program having at least one code section, the at least one code section being executable by a machine comprising at least one processor, for causing the machine to perform one or more steps comprising: acquiring dataset based on a medical imaging technique during medical imaging based examination of a patient; generating one or more medical images based on the acquired dataset; displaying the one or more medical images via a display device; providing an end-result associated with at least one medical image; and providing analysis assistance relating to the end-result associated with the at least one medical image, wherein providing the analysis assistance comprises, automatically: identifying one or more intermediate steps performed or used in obtaining or determining the end-result; determining for each of the one or more intermediate steps corresponding information; and providing to a user the determined information associated with each of the one or more intermediate steps.

In an example implementation, the one or more steps further comprise providing the analysis assistance in response to at least one trigger, wherein the at least one trigger comprises at least one of user input or pre-defined condition.

In an example implementation, the one or more steps further comprise performing at least some of the functions associated with providing the analysis assistance using artificial intelligence (AI) based processing.

In an example implementation, the one or more steps further comprise providing the determined information associated with at least one intermediate step in conjunction with display at least one corresponding image.

In an example implementation, providing the determined information associated with the at least one intermediate step further comprises: generating at least one medical image based on the acquired dataset corresponding to the at least one intermediate step; adjusting the at least one medical image to incorporate at least a portion of the determined information, or visual indication corresponding to the determined information; and displaying the at least one medical image via the display device.

In an example implementation, the one or more steps further comprise providing the end-result associated with the at least one medical image in response to at least one trigger, wherein the at least one trigger comprises a tool that is executed in response to at least one of user input and pre-defined condition.

In an example implementation, the tool comprises an artificial intelligence (AI) based tool.

An example system, for use of analysis assistant, in accordance with the present disclosure, comprises: a scanner configured to obtain imaging signals based on a medical imaging technique; a display device configured to display images; and one or more circuits configured to: generate dataset based on the obtained imaging signals; generate one or more medical images based on the acquired dataset; display the one or more medical images via the display device; provide an end-result associated with at least one medical image; and provide analysis assistance relating to the end-result associated with the at least one medical image, wherein providing the analysis assistance comprises, automatically: identifying one or more intermediate steps performed or used in obtaining or determining the end-result; determining for each of the one or more intermediate steps corresponding information; and providing to a user the determined information associated with each of the one or more intermediate steps.

In an example implementation, the one or more circuits are configured to provide the analysis assistance in response to at least one trigger, wherein the at least one trigger comprises at least one of user input or pre-defined condition.

In an example implementation, the one or more circuits are configured to perform at least some of the functions associated with providing the analysis assistance using artificial intelligence (AI) based processing.

In an example implementation, the one or more circuits are configured to provide the determined information associated with at least one intermediate step in conjunction with display at least one corresponding image.

In an example implementation, the one or more circuits are configured to, when providing the determined information associated with the at least one intermediate step: generate at least one medical image based on the acquired dataset corresponding to the at least one intermediate step; adjust the at least one medical image to incorporate at least a portion of the determined information, or visual indication corresponding to the determined information; and display the at least one medical image via the display device.

In an example implementation, the one or more circuits are configured to provide the end-result associated with the at least one medical image in response to at least one trigger, wherein the at least one trigger comprises a tool that is executed in response to at least one of user input and pre-defined condition.

As utilized herein the terms “circuits” and “circuitry” refer to physical electronic components (e.g., hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. In other words, “x and/or y” means “one or both of x and y.” As another example, “x, y, and/or z” means any element of the seven-element set {(x), (y), (z), (x, y), (x, z), (y, z), (x, y, z)}. In other words, “x, y and/or z” means “one or more of x, y, and z.” As utilized herein, the terms “block” and “module” refer to functions than can be performed by one or more circuits. As utilized herein, the term “exemplary” means serving as a non-limiting example, instance, or illustration. As utilized herein, the terms “for example” and “e.g.,” set off lists of one or more non-limiting examples, instances, or illustrations. As utilized herein, circuitry is “operable” to perform a function whenever the circuitry comprises the necessary hardware (and code, if any is necessary) to perform the function, regardless of whether performance of the function is disabled or not enabled (e.g., by some user-configurable setting, a factory trim, etc.).

Other embodiments of the invention may provide a non-transitory computer readable medium and/or storage medium, and/or a non-transitory machine readable medium and/or storage medium, having stored thereon, a machine code and/or a computer program having at least one code section executable by a machine and/or a computer, thereby causing the machine and/or computer to perform the processes as described herein.

Accordingly, the present disclosure may be realized in hardware, software, or a combination of hardware and software. The present invention may be realized in a centralized fashion in at least one computing system, or in a distributed fashion where different elements are spread across several interconnected computing systems. Any kind of computing system or other apparatus adapted for carrying out the methods described herein is suited. A typical combination of hardware and software may be a general-purpose computing system with a program or other code that, when being loaded and executed, controls the computing system such that it carries out the methods described herein. Another typical implementation may comprise an application specific integrated circuit or chip.

Various embodiments in accordance with the present disclosure may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

What is claimed is:

1. A method comprising:
acquiring dataset based on a medical imaging technique during medical imaging based examination of a patient;
generating one or more medical images based on the acquired dataset;
displaying the one or more medical images via a display device;
providing an end-result associated with at least one medical image; and
providing analysis assistance relating to the end-result associated with the at least one medical image, wherein providing the analysis assistance comprises, automatically:
identifying one or more intermediate steps performed or used in obtaining or determining the end-result;
determining for each of the one or more intermediate steps corresponding information; and
providing to a user the determined information associated with each of the one or more intermediate steps.

2. The method of claim 1, further comprising providing the analysis assistance in response to at least one trigger, wherein the at least one trigger comprises at least one of user input or pre-defined condition.

3. The method of claim 1, further comprising performing at least some of the functions associated with providing the analysis assistance using artificial intelligence (AI) based processing.

4. The method of claim 1, further comprising providing the determined information associated with at least one intermediate step in conjunction with display at least one corresponding image.

5. The method of claim 4, wherein providing the determined information associated with the at least one intermediate step further comprises:
generating at least one medical image based on the acquired dataset corresponding to the at least one intermediate step;
adjusting the at least one medical image to incorporate at least a portion of the determined information, or visual indication corresponding to the determined information; and
displaying the at least one medical image via the display device.

6. The method of claim 1, further comprising providing the end-result associated with the at least one medical image in response to at least one trigger, wherein the at least one trigger comprises a tool that is executed in response to at least one of user input and pre-defined condition.

7. The method of claim 6, wherein the tool comprises an artificial intelligence (AI) based tool.

8. A non-transitory computer readable medium having stored thereon a computer program having at least one code section, the at least one code section being executable by a machine comprising at least one processor, for causing the machine to perform one or more steps comprising:
acquiring dataset based on a medical imaging technique during medical imaging based examination of a patient;
generating one or more medical images based on the acquired dataset;
displaying the one or more medical images via a display device;
providing an end-result associated with at least one medical image; and
providing analysis assistance relating to the end-result associated with the at least one medical image, wherein providing the analysis assistance comprises, automatically:
identifying one or more intermediate steps performed or used in obtaining or determining the end-result;
determining for each of the one or more intermediate steps corresponding information; and
providing to a user the determined information associated with each of the one or more intermediate steps.

9. The non-transitory computer readable medium of claim 8, further comprising providing the analysis assistance in response to at least one trigger, wherein the at least one trigger comprises at least one of user input or pre-defined condition.

10. The non-transitory computer readable medium of claim 8, further comprising performing at least some of the functions associated with providing the analysis assistance using artificial intelligence (AI) based processing.

11. The non-transitory computer readable medium of claim 8, further comprising providing the determined information associated with at least one intermediate step in conjunction with display at least one corresponding image.

12. The non-transitory computer readable medium of claim 12, wherein providing the determined information associated with the at least one intermediate step further comprises:
generating at least one medical image based on the acquired dataset corresponding to the at least one intermediate step;
adjusting the at least one medical image to incorporate at least a portion of the determined information, or visual indication corresponding to the determined information; and
displaying the at least one medical image via the display device.

13. The non-transitory computer readable medium of claim 8, further comprising providing the end-result associated with the at least one medical image in response to at least one trigger, wherein the at least one trigger comprises a tool that is executed in response to at least one of user input and pre-defined condition.

14. The non-transitory computer readable medium of claim 14, wherein the tool comprises an artificial intelligence (AI) based tool.

15. A system comprising:
a scanner configured to obtain imaging signals based on a medical imaging technique;
a display device configured to display images; and
one or more circuits configured to:
generate dataset based on the obtained imaging signals;
generate one or more medical images based on the acquired dataset;
display the one or more medical images via the display device;
provide an end-result associated with at least one medical image; and
provide analysis assistance relating to the end-result associated with the at least one medical image, wherein providing the analysis assistance comprises, automatically:
identifying one or more intermediate steps performed or used in obtaining or determining the end-result;
determining for each of the one or more intermediate steps corresponding information; and
providing to a user the determined information associated with each of the one or more intermediate steps.

16. The system of claim 15, wherein the one or more circuits are configured to provide the analysis assistance in response to at least one trigger, wherein the at least one trigger comprises at least one of user input or pre-defined condition.

17. The system of claim 15, wherein the one or more circuits are configured to perform at least some of the functions associated with providing the analysis assistance using artificial intelligence (AI) based processing.

18. The system of claim 15, wherein the one or more circuits are configured to provide the determined information associated with at least one intermediate step in conjunction with display at least one corresponding image.

19. The system of claim 18, wherein the one or more circuits are configured to, when providing the determined information associated with the at least one intermediate step:
generate at least one medical image based on the acquired dataset corresponding to the at least one intermediate step;
adjust the at least one medical image to incorporate at least a portion of the determined information, or visual indication corresponding to the determined information; and
display the at least one medical image via the display device.

20. The system of claim 15, wherein the one or more circuits are configured to provide the end-result associated with the at least one medical image in response to at least one trigger, wherein the at least one trigger comprises a tool that is executed in response to at least one of user input and pre-defined condition.
